(19) United States
(12) Patent Application Publication
(10) Pub. No.: US 2025/0009523 A1
(43) Pub. Date: Jan. 9, 2025

(54) NON-NEWTONIAN FLUID-BASED SPINAL DISC REPLACEMENT DEVICE AND METHODS THEREOF

(71) Applicant: Timothy Tian Roberts, New York, NY (US)

(72) Inventor: Timothy Tian Roberts, New York, NY (US)

(21) Appl. No.: 18/204,936

(22) Filed: Jun. 1, 2023

Related U.S. Application Data
(60) Provisional application No. 63/347,992, filed on Jun. 1, 2022.

(51) Int. Cl. A61F 2/44; A61F 2/46; A61F 2/30

(52) U.S. Cl. CPC A61F 2/441; A61F 2/4611; A61F 2002/30242; A61F 2002/443; A61F 2002/444

(57) ABSTRACT

A spinal disc replacement device includes a first balloon configured for insertion in a cavity within an intervertebral disc (IVD), the cavity surrounded by an annulus fibrosis (AF). In some embodiments, the spinal disc replacement device further includes a first material disposed within the first balloon, where the first material includes a first non-Newtonian fluid (NNF) and provides an NNF-filled balloon within the cavity. In some examples, the NNF-filled balloon is configured to support vertebrae disposed above the IVD.

### CROSS-REFERENCE TO RELATED APPLICATION

This application claims the benefit of U.S. Provisional Application No. 63/347,992, filed Jun. 1, 2022, the entirety of which is incorporated by reference herein.

### BACKGROUND

Diseases of the spinal intervertebral disc are extremely common and are the leading cause of back pain and back pain-related disability in the US. Severe cervical (neck) and lumbar (low back) disc disease is often treated with spinal fusion, or arthrodesis, which includes the surgical removal of the intervertebral disc followed by implantation of various bone graft materials, mechanical spacers, and/or screw and rod fasteners which essentially hold the vertebral bones in position until they solidify (fuse) together. While joint replacement, or arthroplasty, is widely used to treat severe joint disease (e.g., such as of the hip and knee), spinal arthrodesis remains the gold-standard treatment for joint (i.e., disc) diseases of the human spine.

In an effort to preserve natural motion of the spine, it is likely that the future of spinal surgery will trend toward adoption of disc arthroplasty. To that end, there have been some attempts at developing effective disc arthroplasty systems including both total disc replacements and partial disc replacements. In an example of partial disc replacement, the soft and viscous center of the disc or the nucleus pulposus (NP), may be replaced while the tough other rim of the disc or annulus fibrosis (AF) remains largely intact. Such partial disc replacement may be generally less invasive, less expensive, and offer faster recovery times as compared to total disc replacement. However, in some cases, NP replacement devices may shift out of position (e.g., migrate) with repetitive loads, no longer supporting vertebral bones and in some cases compressing nearby nerve root structure. In addition, the implant material used in NP replacement devices may become brittle and inelastic, thus no longer facilitating natural spine motion.

Thus, existing techniques have not proved entirely satisfactory in all respects.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 illustrates a lateral view of a portion of vertebrae of a human spine, in accordance with some embodiments;

FIG. 2 illustrates a lateral view of the portion of vertebrae of FIG. 1 upon insertion of a needle or other insertion device, in accordance with some embodiments;

FIG. 3 illustrates a lateral view of the portion of vertebrae of FIG. 1 upon removal of a first portion of the nucleus pulposus (NP) of an intervertebral disc (IVD) and formation of a cavity within the IVD, in accordance with some embodiments;

FIG. 4 illustrates a lateral view of the portion of vertebrae of FIG. 1 upon removal of a second portion of the NP of the IVD and expansion of the cavity within the IVD, in accordance with some embodiments;

FIG. 5 illustrates a lateral view of the portion of vertebrae of FIG. 1 upon removal of substantially all of the NP of the IVD, further expansion of the cavity within the IVD, and insertion of a balloon, in accordance with some embodiments;

FIG. 6 illustrates a lateral view of the portion of vertebrae of FIG. 1 while using a non-Newtonian fluid (NNF) to begin to fill the balloon, in accordance with some embodiments;

FIG. 7 illustrates a lateral view of the portion of vertebrae of FIG. 1 while using the NNF to further fill the balloon, in accordance with some embodiments;

FIG. 8 illustrates a lateral view of the portion of vertebrae of FIG. 1 after filling the balloon with the NNF, in accordance with some embodiments;

FIG. 9 illustrates a lateral view of the portion of vertebrae of FIG. 1 upon insertion of a secondary needle or other insertion device into the NNF-filled balloon, in accordance with some embodiments;

FIG. 10 illustrates a lateral view of the portion of vertebrae of FIG. 1 upon insertion of a secondary balloon within the NNF-filled balloon, in accordance with some embodiments;

FIG. 11 illustrates a lateral view of the portion of vertebrae of FIG. 1 after filling the secondary balloon with a hard cement, in accordance with some embodiments;

FIG. 12 illustrates a lateral view of the portion of vertebrae of FIG. 1 after removal of the secondary needle or other insertion device, in accordance with some embodiments;

FIGS. 13, 14, 15, 16, 17, 18, 19, 20, and 21 illustrate alternative views of forming a balloon-in-a-balloon assembly within a cavity of an IVD, in accordance with some embodiments;

FIG. 22 illustrates a side view of an embodiment of an NNF-based total disc replacement device, in accordance with some embodiments;

FIG. 23 illustrates a lateral view of a portion of vertebrae of a human spine including the NNF-based total disc replacement device of FIG. 22, in accordance with some embodiments; and

FIG. 24 is a flow chart of a method of forming an NNF-based NP replacement device, according to one or more aspects of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, a first element coupled to a second element in the description that follows may include embodiments in which the first and second elements are directly coupled such that they are in direct contact, and may also include embodiments in which additional elements may be coupled between the first and second elements, such that the first and second elements may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Still further, when a number or a range of numbers is described with “about,” “approximate,” and the like, the term is intended to encompass numbers that are within a reasonable range including the number described, such as within +/−10% of the number described or other values as understood by person skilled in the art. For example, the term “about 1 mm” encompasses the dimension range from 0.9 mm to 1.1 mm.

The present disclosure generally relates to a spinal intervertebral disc (IVD) replacement device, such as a non-Newtonian fluid (NNF)-based IVD replacement device, as described in more detail below. The spinal IVD is the principal joint between adjacent vertebrae along the lumbar, thoracic, and cervical spine, and it is vital to the normal functioning of the human spine. Generally, IVDs provide flexibility, stability, cushioning (e.g., to provide a shock-absorbing effect within the spine and prevent vertebrae from grinding together), and help to protect the nerves that run down the spine and between the vertebrae. Intervertebral disc disease can be characterized by the breakdown (e.g., degeneration) of one or more IVDs. While often affecting discs in the lumbar region of the spine, discs within any portion of the spine (e.g., lumbar, thoracic, or cervical spine) may be affected by intervertebral disc disease.

In particular, diseases of the spinal intervertebral disc are extremely common and are the leading cause of back pain and back pain-related disability in the US. In 2013, as one example, there were more than 57 million physician visits in the US for complaints of back pain alone. In various existing implementations, severe cervical (neck) and lumbar (low back) disc disease is often treated with spinal fusion, or the surgical removal of the intervertebral disc followed by implantation of various bone graft materials, mechanical spacers, and/or screw and rod fasteners which essentially hold the vertebral bones in position until they solidify (fuse) together. This technique, termed arthrodesis (Greek: arthro—joint; -desis—binding) was employed in the earlier half of the 20^{th} century for a variety of severe joint diseases, including severe hip and knee arthritis. Patients undergoing hip or knee arthrodesis would have a fixed joint that would allow a painless and stable, albeit unnatural, gait and posture. Several mid-20^{th} century innovations in the field of joint replacement, termed arthroplasty (-plasty from the Greek plastos, meaning to mold), led to the widespread adoption of hip and knee replacements for severe joint disease. Today, joint arthroplasty is performed by the hundreds of thousands in the US annually and is the gold-standard treatment for a variety of joint conditions.

Despite these breakthroughs in joint replacement, spinal arthrodesis remains the gold-standard treatment for joint (i.e., disc) diseases of the human spine. Adoption and development of spinal arthroplasty lags considerably behind those of the hip and knee. Reasons for the dominance of spinal fusion are multidimensional and include the relatively complex biomechanics of the spine (e.g., as compared to a mechanically simple ball-and-socket hip joint), technical surgical challenges of implanting such devices, concerns over the biocompatibility of certain implant materials, economic incentives of fusion, and the fact that spinal fusion has relatively high patient satisfaction scores when compared to fusions of larger and less redundant joints. Ultimately, however, many believe that the future of surgery for spinal disc disease will trend toward preserving natural motion through disc arthroplasty.

In the past two decades, there have been several notable attempts at developing effective disc arthroplasty systems. Disc arthroplasty devices fall into two categories: total disc replacements and partial disc replacements. Total disc replacements replace both the soft and viscous center of the disc, known as the nucleus pulposus (NP) as well as the outer, tougher rim of the disc, known as the annulus fibrosis (AF). While these devices have had some success in the cervical spine, they have fallen far from mainstream adoption in the lumbar and thoracic spine. Alternative to total disc replacements are partial disc replacements, and several devices have been made or proposed to replace only the soft and viscous NP portion of the disc. Replacement of the NP alone may lend itself to less invasive, perhaps endoscopic (e.g., with a camera) or percutaneous (e.g., through needles, typically with X-ray or ultrasound guidance) insertion techniques. Implantation of these smaller devices, which may not be as rigid or cumbersome as the larger solid metal and/or plastic total disc replacement systems, may be associated with smaller and less expensive procedures, shorter operative and hospital times, and faster recoveries.

While partial disc replacements would appear to offer several advantages over total disc replacements, they are not routinely performed in the US and are considered essentially experimental at this time. Nevertheless, several attempts have been made at nucleus replacement, although none of the proposed devices have thus far had significant commercial or clinical success. One exemplary NP replacement device, the PDN (Prosthetic Disc Nucleus; Raymedica, Inc. of Bloomington, MN) achieved some short-term clinical success, but devices ultimately failed for two key reasons. First, implants often migrated from their intended positions with repetitive loads. Once out of position, the devices no longer supported the vertebral bones and, worse, sometimes came to compress nearby nervous or other critical structures. Second, in a case report detailing the surgical removal and evaluation of a failed PDN implant, the implant material had become brittle and inelastic and was no longer facilitating natural spinal motion. Thus, existing disc replacement devices have not proved entirely satisfactory in all respects.

Embodiments of the present disclosure offer advantages over the existing art, though it is understood that other embodiments may offer different advantages, not all advantages are necessarily discussed herein, and no particular advantage is required for all embodiments. For example, embodiments of the present disclosure provide an improved disc replacement device and related method that effectively addresses the shortcomings of existing disc replacement devices, such as those described above. In various embodiments, the disclosed disc replacement device includes a non-Newtonian fluid (NNF)-based NP replacement device. Further, in some cases, the disclosed disc replacement device may include a partial disc replacement device or a total disc replacement device, as discussed in more detail below.

A non-Newtonian fluid (NNF) is a fluid whose behavior deviates from Newton's law of viscosity, i.e., liquids that maintain a constant viscosity independent of stresses applied to them. In other words, Newtonian fluids, like water, tend to stay liquid whether still, flowing, or under relative pressure. For non-Newtonian fluids (NNFs), viscosity can change as forces are applied to the fluid. For example, the fluid may become runnier under pressure or stiffer under pressure. In general, an NNF may include one of a dilatant NNF (viscosity increases when force is applied), a pseudoplastic NNF (viscosity decreases when force is applied), a rheopectic NNF (viscosity increases with applied force and over time), or a thixotropic NNF (viscosity decreases with applied force and over time). A classic example of an NNF (e.g., a dilatant NNF) is a cornstarch-water suspension, colloquially known as Oobleck. When large forces are applied, the liquid becomes firm and rigidly holds its shape. Alternatively, when weaker forces are applied, it flows like a viscous liquid. A pool full of Oobleck, for example, can provide an immersive bath when the subject is at rest, but can be traversed on foot (e.g., ran across) if the subject steps quickly enough upon its surface. In some cases, an NNF may include a combination of a stable polysaccharide and a solvent (e.g., such as alcohol or other suitable solvent), polyvinyl alcohol-based glues and borax (sometimes referred to “slime” or “goop”), a clay/silt colloid suspension in saline (a variant of quicksand), or other appropriate composition.

NP replacement using an NNF-based NP replacement device, in accordance with the various embodiments disclosed herein, may offer several significant advantages over existing NP replacement devices. For instance, NNF-based NP replacement devices are amenable to percutaneous (needle or very small tube) insertion or injection into an intervertebral disc space. An NNF, in some examples, could be freely injected into the inner disc space if the outer rim of the disc (AF) is intact to contain it. Alternatively, in some cases, the NNF may be inserted through a balloon-catheter type device. In such a balloon insertion method/device, a needle can be placed into the disc space through which remnants of the NP may be aspirated (suctioned out) or removed with various manual graspers and/or motorized shavers. Afterwards, an elastic balloon of sufficient thickness may be inserted into a cavity within the disc space that was created when the NP was removed. In some embodiments, the balloon may include an empty pre-sealed elastic shell into which the NNF is subsequently injected with a piercing needle. Alternatively, in some cases, the balloon may have an opening through which the NNF may be injected, and which is subsequently sealed and detached from the balloon insertion device. In various embodiments, the balloon may be filled using an appropriate volume of the NNF to sufficiently fill the NP void (cavity) and sufficiently support the vertebrae above. After filling the balloon with the NNF, the injection needle may be removed from the balloon, which henceforth contains the NNF, or the balloon is then sealed and detached from the cannula. Thereafter, the cannula/tube/needle may be removed while the NNF-filled balloon remains within the inner disc space.

The benefits of an NNF-based NP replacement, as discussed, may overcome several shortcomings associated with existing NP replacements. For example, in some embodiments and because the NNF is contained within a sealed and impermeable balloon, the NNF inside the balloon would be shielded from the potentially degradative effects of the body's milieu. Some existing elastomeric devices, such as the PDN, were not coated or insulated and became markedly stiffer once exposed to the in vivo environment. In addition, the NNF-based NP replacement would be resistant to migration, the other major mode of failure for at least some existing NP replacements. In accordance with the embodiments disclosed herein, migration would be mitigated in two distinct ways. First, because of the minimally invasive insertion techniques, large access “windows” within the AF (the thick fibrous skin surrounding the relatively soft NP) would need not be formed (e.g., by removing or disrupting large portions of the AF). This would provide a secure and more physiological/natural constraint for the NNF-filled balloon. Second, in accordance with the non-Newtonian properties of the fluid, when subjected to intense and/or rapid forces, such as compression pressure from jumping, jogging, or falling, the NNF material inside the NNF-filled balloon would become firm and hold instantaneously static in shape, thereby (i) resisting migration and (ii) providing a relatively wide surface area across which the compression forces would be experienced or spread. This would also provide resistance to device subsidence or endplate fracture.

Further, in some embodiments, the NNF-based NP replacement device or implant described herein may be used in conjunction with a rigid, solid device, similar to a Fernstrom ball bearing. The Fernstrom device, first implanted in the mid-20^{th} century, included a round steel ball placed between the vertebral bones. As a round bearing, it allowed the vertebrae to flex, extend, lean to the right and left, and rotate left and right. The technique was initially promising, but implants soon failed because the extremely hard steel ball (far harder than human bone), with its focal pressure point on the vertebrae plate above, would inevitably end up fracturing the bone. This caused the implant to subside, or sink, into the vertebral bodies, leading to collapse of the intervertebral space and therefore loss of motion. While gentle everyday movements did not necessarily break the bony endplates, sudden and forceful compression could. By combining a device like the Fernstrom ball with a NNF NP, it may be possible to combine the optimal structural support or separation of the vertebral bodies afforded by the hard ball bearing, while the surrounding NNF shields the vertebral bony endplates from excessive point loading when sudden forceful loading occurs. It is noted, however, that while a relatively large steel ball bearing would be difficult to insert through a minimally invasive cannula or needle-insertion device, a balloon within or alongside the NNF NP may be filled with a hard bone cement such as polymethylmethacrylate (PMMA), calcium phosphate cements (CPCs), glass ionomer cements (GICs), or a comparatively firmer substance with greater resistance to compression, which would be injected as a liquid but would harden upon injection. Such a secondary comparatively solid spacer might comprise a variety of shapes or compositions, so long as it served to space the vertebral bones an appropriate distance apart while allowing for physiological or near-physiological motion.

In addition, the safe in vivo use of starch polymers, including cornstarch, has been well established in the literature. Minimal tissue toxicity is anticipated in the event the NNF-filled balloon should rupture. This is opposed, for example, to some prior attempts which used vulcanized rubber as an NP replacement, the byproducts of which were demonstrated to be carcinogenic. Those skilled in the art will recognize other benefits and advantages of the methods and disc replacement device as described herein, and the embodiments described are not meant to be limiting beyond what is specifically recited in the claims that follow.

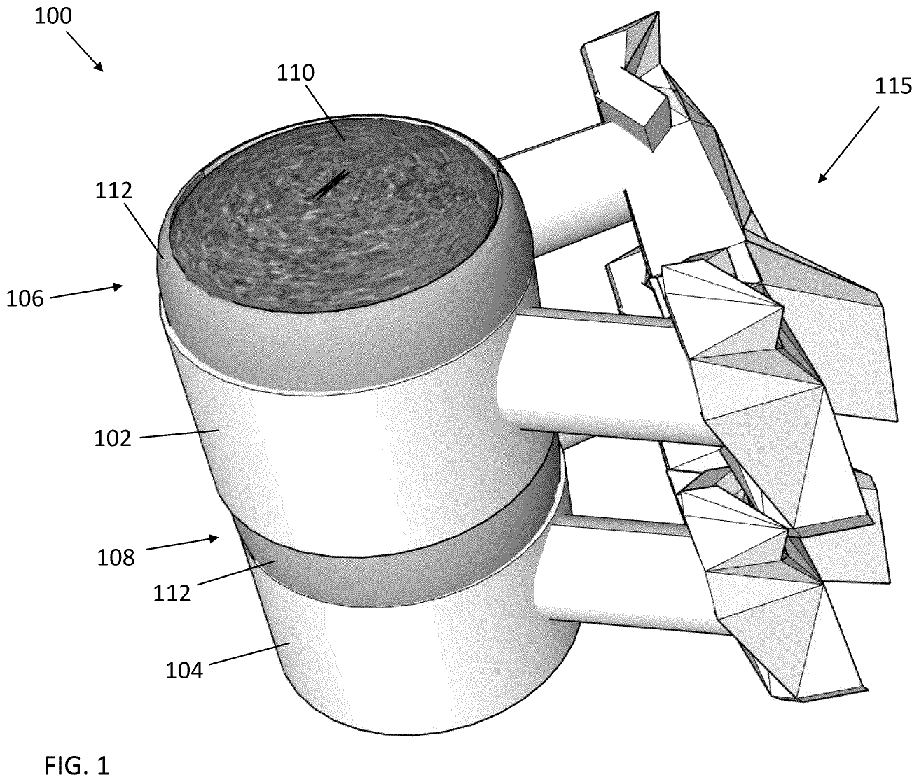

Referring now to FIG. 1, illustrated therein is a lateral view of a portion of vertebrae 100 of a human spine, in accordance with some embodiments. In various embodiments, the portion of vertebrae 100 may be part of the lumbar, thoracic, or cervical spine. In particular, the illustrated portion 100 includes vertebral bodies 102, 104 and intervertebral discs 106, 108. A posterior region 115 of the portion of vertebrae 100, including for example the spinous process and the transverse process, is also illustrated. Each of the intervertebral discs 106, 108 is composed of a nucleus pulposus (NP) 110 (the soft and viscous center of the disc) and an annulus fibrosis (AF) 112 (the tougher, outer rim of the disc). As discussed further herein, an NNF may be used as a replacement for some or all of the NP 110 for one or more intervertebral discs (e.g., such as the intervertebral discs 106, 108, or others). For purposes of the discussion that follows, the NP 110 of the intervertebral disc 106 is replaced by the NNF (or NNF-based NP replacement device).

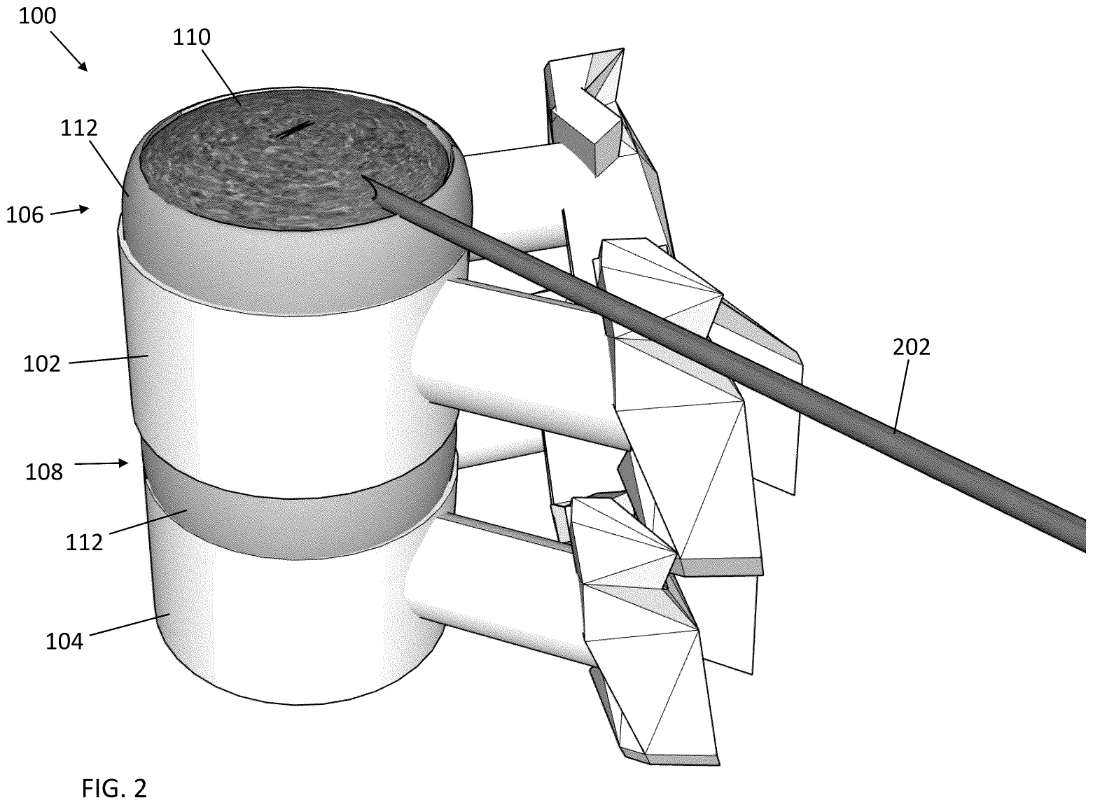

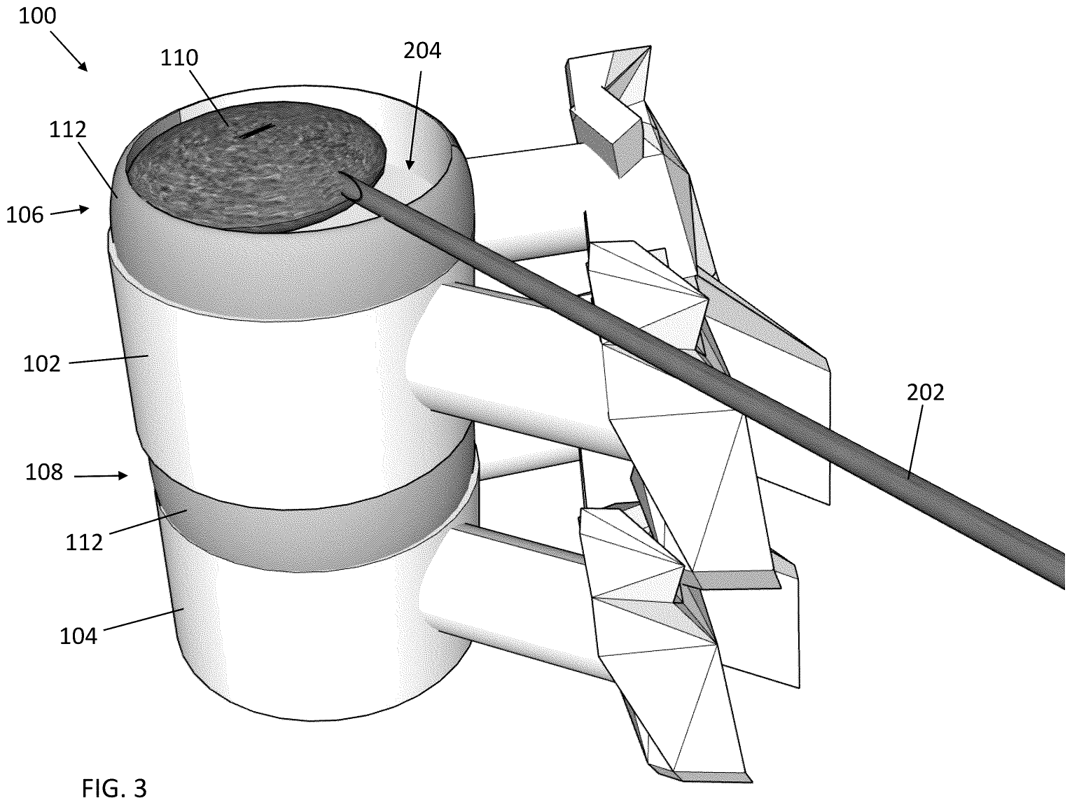

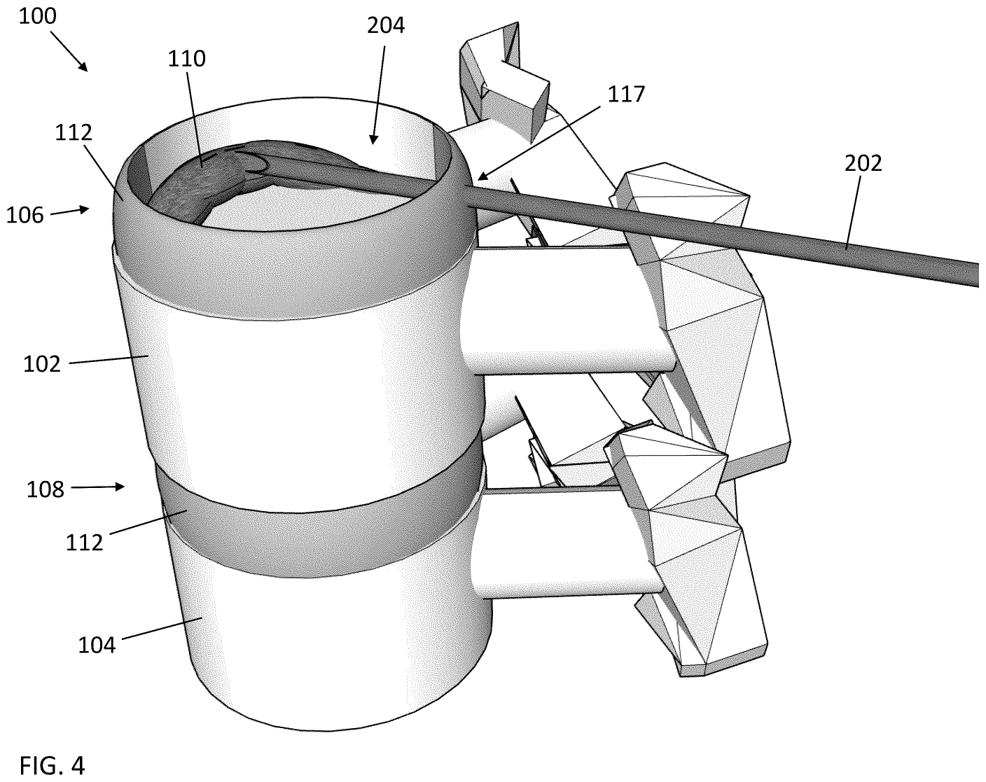

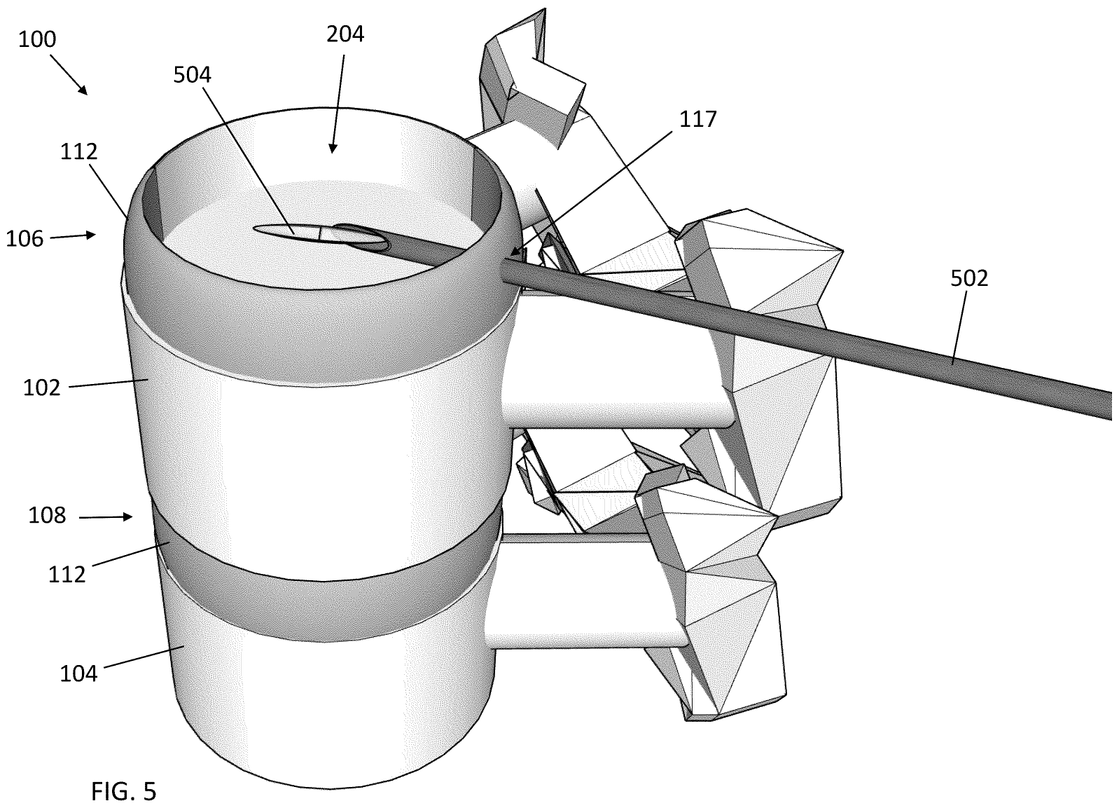

With reference to FIG. 2, a small hole 117 may be formed in the AF 112 (e.g., using a biopsy punch) through which a needle/cannula 202 is inserted. In some embodiments, the hole 117 may have a diameter of about 2.5 mm. In other cases, the hole 117 may have a diameter of between about 2 mm and 3 mm, however it will be understood that other diameters may also be used. Alternatively, the insertion device might be a blunt cannula which divides rather than cuts the annulus fibrosis 112. Insertion of the needle 202 through the hole 117 in the AF 112 is better illustrated, for example, in FIGS. 4 and 5. The inserted needle 202 may then be used to aspirate some or all of the NP 110. Alternatively, various manual graspers, rongeurs, and/or motorized shavers may be used to extract some or all of the NP 110 through the small hole 117 in the AF 112. The example of FIG. 2 illustrates the intact NP 110 upon insertion of the needle 202. In the example of FIG. 3, a portion of the NP 110 has been removed/aspirated, thereby creating a cavity 204 within the intervertebral disc 106. In the example of FIG. 4, an additional portion of the NP 110 has been removed/aspirated, thereby increasing a size of the cavity 204 within the intervertebral disc 106. In the example of FIG. 5, substantially all of the NP 110 has been removed/aspirated, further increasing the size of the cavity 204. In an example, if substantially all of the NP 110 has been removed, then the size of the cavity 204 may be substantially equal to a volume enclosed by the AF 112, the vertebral body 102 below, and another vertebral body above (not shown). For purposes of the present example, it will be assumed that substantially all of the NP 110 has been removed/aspirated, and the replacement NNF (or NNF-based NP replacement device) may substantially fill an entirety of the space previously occupied by the NP 110. However, it is noted that in some embodiments, for example, when only a first portion of the NP 110 is removed, the replacement NNF (or NNF-based NP replacement device) may again be used to substantially fill an entirety of the space previously occupied by the first portion of the NP 110, while a second (unremoved) portion of the NP 110 remains within the intervertebral disc 106.

In some embodiments, after partial or complete removal of the NP 110, an NNF may be freely injected into cavity 204 (e.g., using the needle/cannula 202 or a different, subsequently inserted needle/cannula), with the surrounding AF 112, the vertebral body 102 below, and another vertebral body above (not shown) serving to contain the NNF. In some examples, after injection of the NNF into the cavity 204 and removal of the needle/cannula, the small hole 117 in the AF 112 may be closed or sealed, or simply left to close on its own.

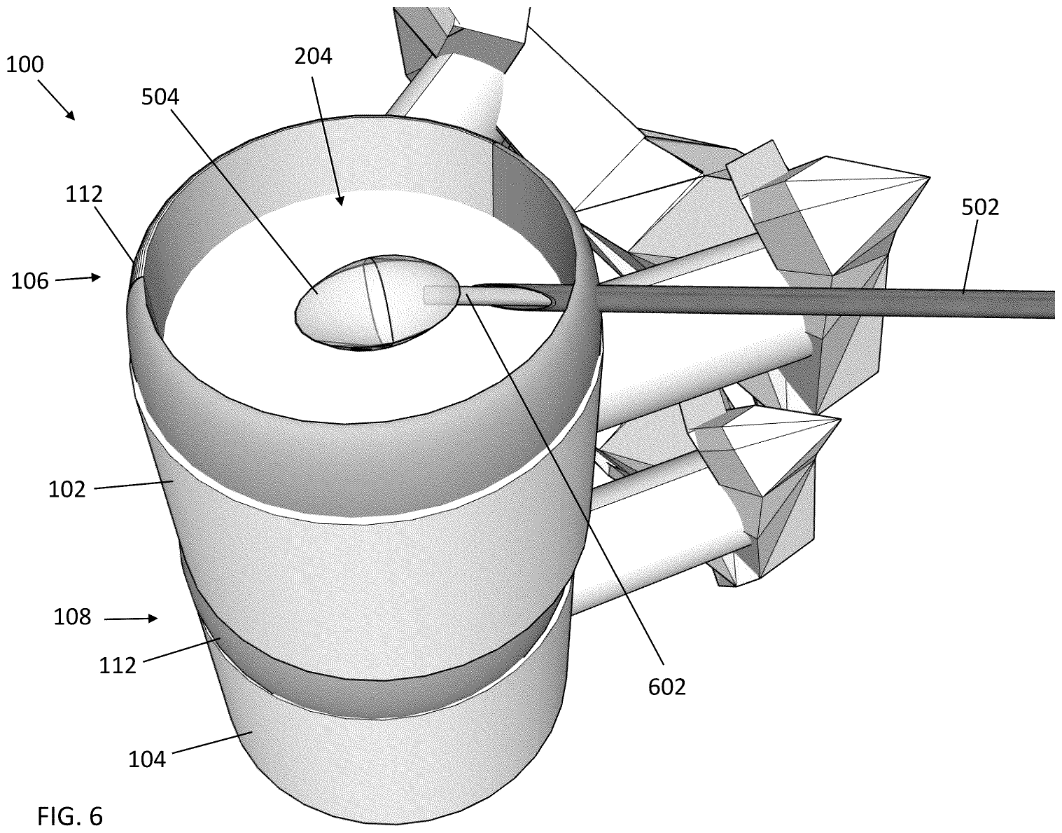

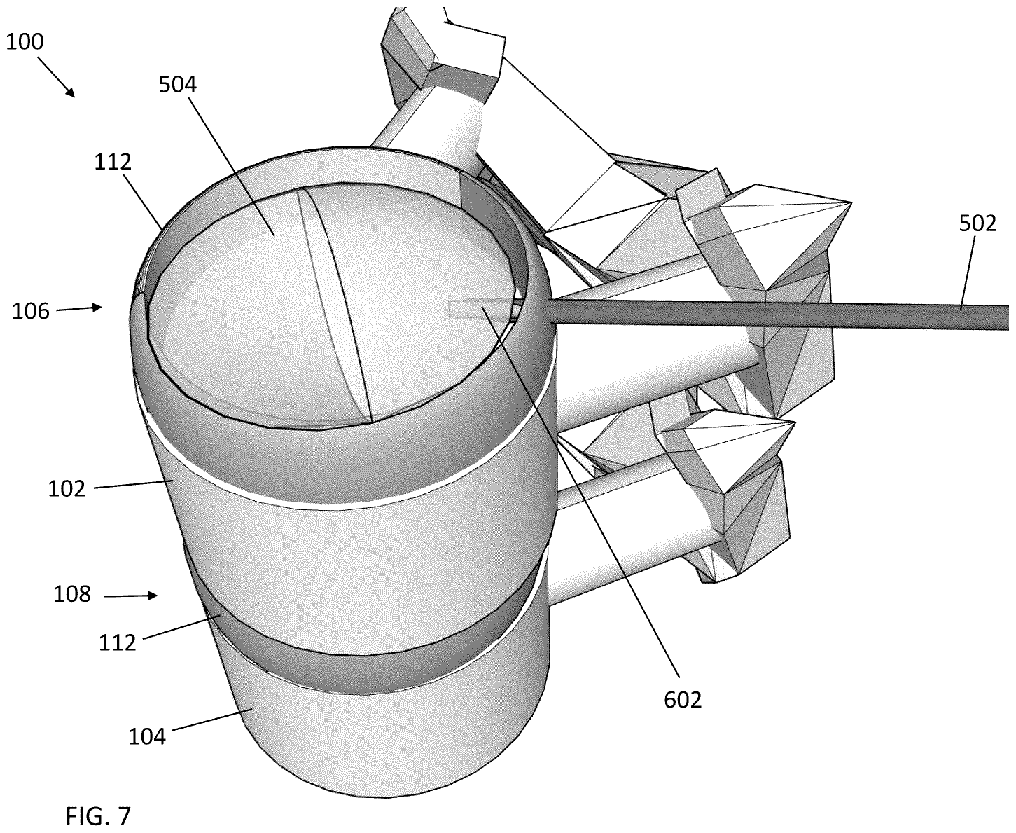

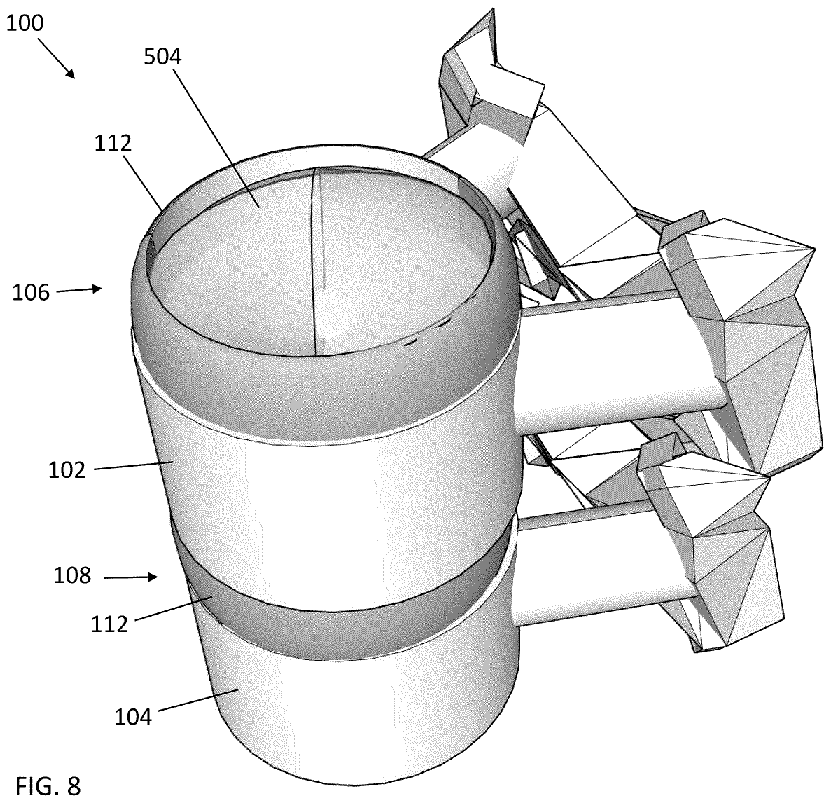

Alternatively, in some embodiments, after partial or complete removal of the NP 110, a balloon insertion device may be used to introduce the NNF into the cavity 204, thereby providing an NNF-based NP replacement device. For purposes of this discussion, it is assumed that a substantial entirety of the NP 110 has been removed/aspirated. After removal of the NP 110, and referring again to FIG. 5, a balloon 504 is inserted into the cavity 204 via a needle/cannula 502. In some cases, the needle/cannula 502 may be the same as the needle/cannula 202 discussed above, or they may be different. The needle/cannula 502 may, in some examples, be referred to as the balloon insertion device. As shown in FIG. 5, the balloon 504 is initially deflated or folded. In some cases, upon insertion of the balloon 504 into the cavity 204 and ejection of the balloon 504 from the needle/cannula 502, the balloon 504 may at least slightly expand/unfold. In various embodiments, the balloon 504 may include an empty pre-sealed elastic shell into which the NNF is subsequently injected with a piercing needle. Alternatively, in some cases, the balloon 504 may have an opening through which the NNF may be injected, and which is subsequently sealed and detached from the balloon insertion device (e.g., such as the needle/cannula 502). After insertion of the balloon 504, and in the example shown in FIG. 6, a needle 602 is inserted through the needle/cannula 502 to pierce the balloon 504. After piercing the balloon 504, the needle 602 may be used to inject the NNF into the balloon 504 and begin filling the balloon 504 with the NNF. In the example of FIG. 7, the balloon 504 has been substantially filled with an NNF such that the balloon 504 substantially occupies an entirety of the cavity 204 (e.g., the space previously occupied by the NP 110). To be sure, in some embodiments, the NNF-filled balloon 504 may occupy less than an entirety of the cavity 204. For instance, it may be desirable in some cases to slightly underfill the balloon 504 in order to subsequently form a second (solid) inner balloon that acts as a solid spacer (e.g., similar to a Fernstrom ball), as discussed in more detail below. It will also be understood, for instance when less than all of the NP 110 has been removed/aspirated, the NNF-filled balloon 504 may still be used to substantially fill an entirety of the (albeit smaller) cavity 204 (e.g., the space previously occupied by a first portion of the NP 110 that was removed, while a second unremoved portion of the NP 110 remains within the intervertebral disc 106). After filling the balloon 504 with the NNF, the needle 602/cannula 502 may be removed, for example, as shown in FIG. 8. In some cases, the balloon 504 may be made of a thick rubber or similar material that seals once the needle 602/cannula 502 is retracted. In some examples, the balloon 504 may be sealed and detached from the needle 602/cannula 502. In some embodiments, after filling the balloon 504 and removing of the needle 602/cannula 502, the small hole 117 in the AF 112 may be closed or sealed, or simply left to close on its own. Whether the small hole 117 in the AF 112 is sealed or not, the NNF-filled balloon 504 remains within the inner disc space (e.g., the cavity 204) and provides sufficient support to the vertebrae above. It is also noted that in various embodiments the shape of the NNF-filled balloon 504 may be roughly discoid or oblate spheroidal, or may be relatively plastic, allowing it to substantially conform to an arbitrary cavity shape (e.g., the arbitrary cavity shape depending in part on an amount of the NP 110 that is removed/aspirated).

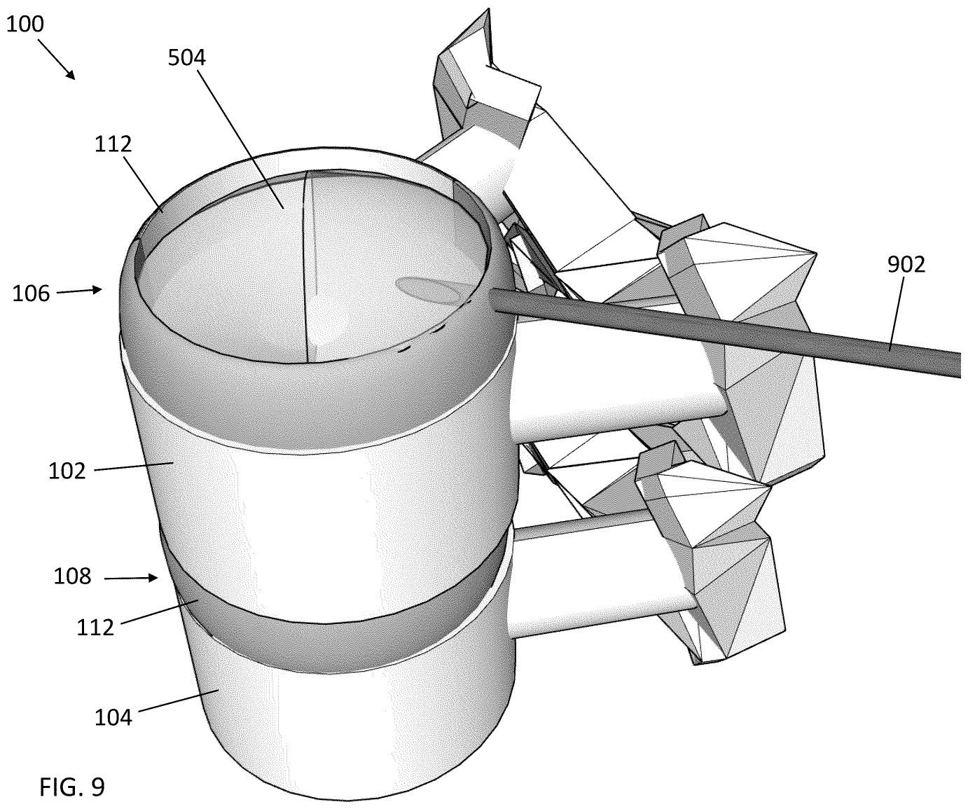

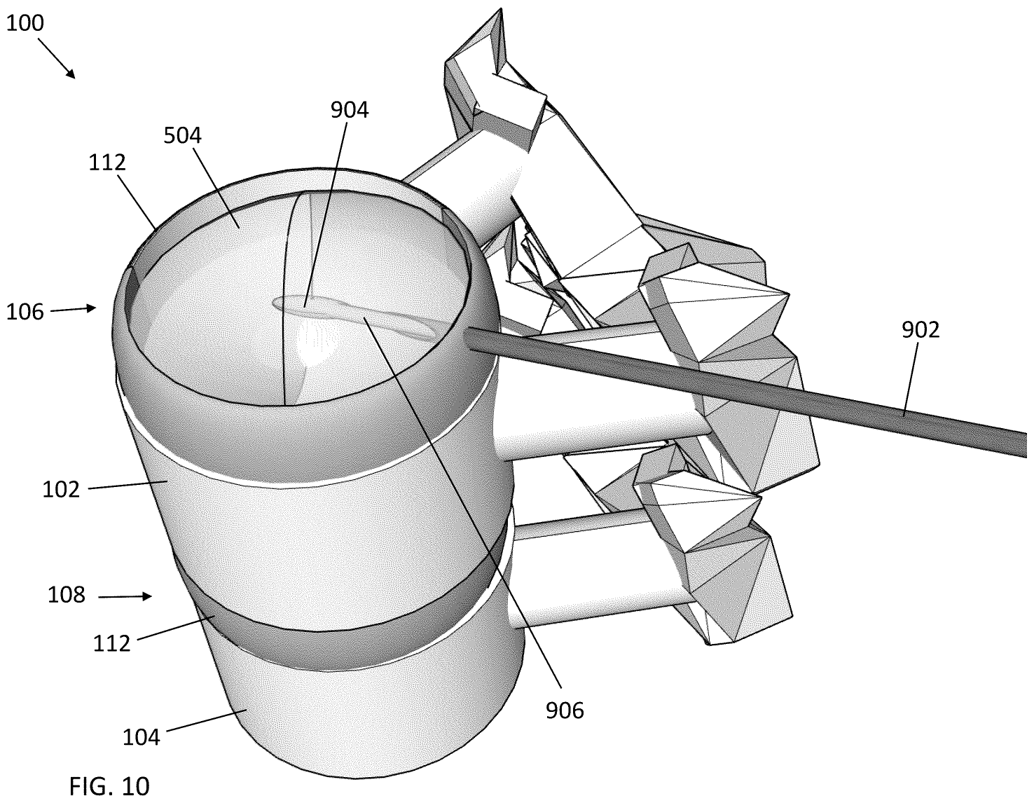

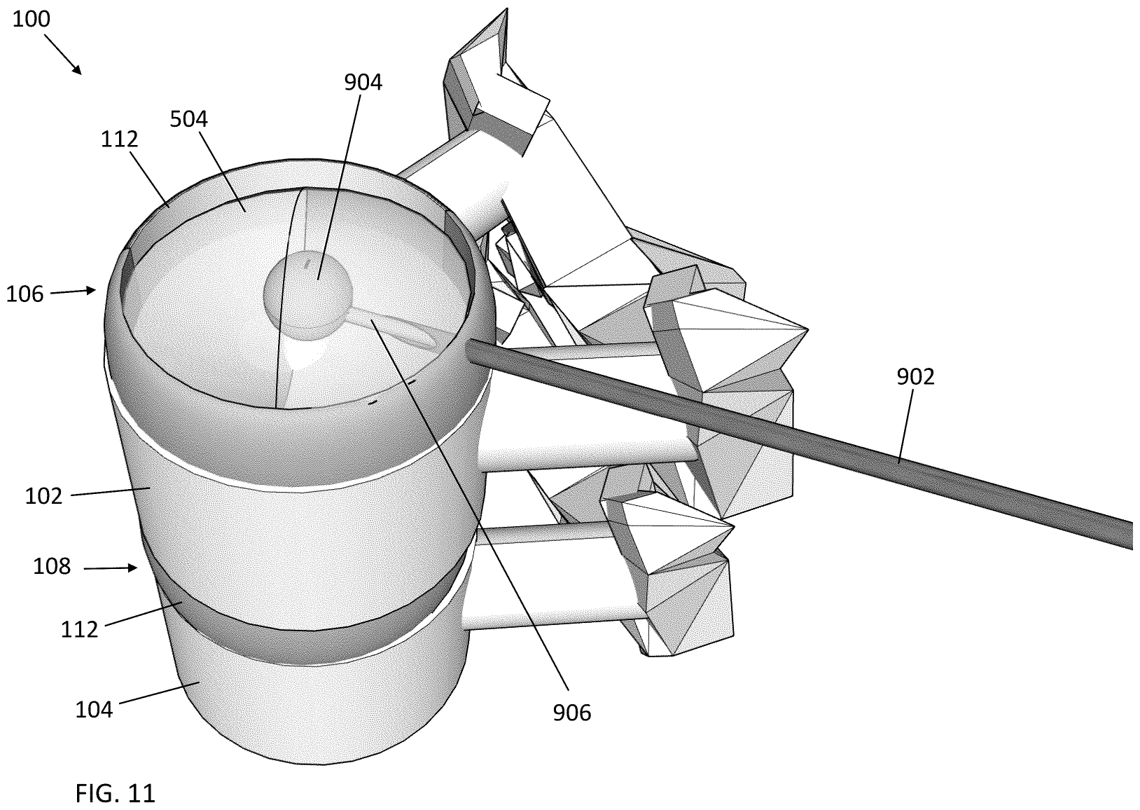

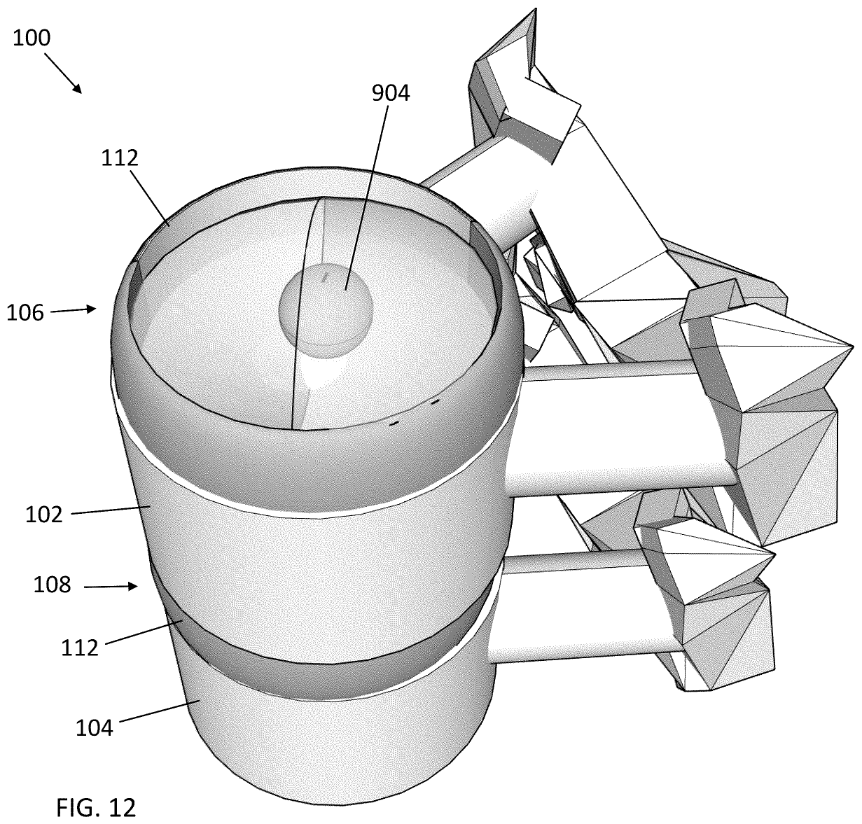

Referring now to the example of FIG. 9, and in some embodiments, after insertion of the NNF-filled balloon 504, a needle/cannula 902 is inserted into the NNF-filled balloon 504 via the previously formed small hole 117 in the AF 112. In some cases, the needle/cannula 902 may be the same as the needle/cannula 502 or 202 discussed above, or they may be different. Thereafter, as shown in FIG. 10, a balloon 904 is inserted into the NNF-filled balloon 504 via the needle/cannula 902. The needle/cannula 902 may, in some examples, also be referred to as a balloon insertion device. In some embodiments, the balloon 904 is initially deflated or folded. In some cases, upon insertion of the balloon 904 into the NNF-filled balloon 504 and ejection of the balloon 904 from the needle/cannula 902, the balloon 904 may at least slightly expand/unfold. In some cases, the balloon 904 may include an empty pre-sealed elastic shell into which a hard cement (e.g., such as PMMA or “bone cement”) is subsequently injected with a piercing needle. Alternatively, in some cases, the balloon 904 may have an opening through which the hard cement may be injected, and which is subsequently sealed and detached from the balloon insertion device (e.g., such as the needle/cannula 902). After insertion of the balloon 904, and in the example shown in FIG. 10, a needle 906 is inserted through the cannula 902 to pierce the balloon 904. After piercing the balloon 904, the needle 906 may be used to inject the hard cement (e.g., initially in liquid form) into the balloon 904 and begin filling the balloon 904. In some cases, the needle 906 and the balloon 904 may be attached (e.g., such as by the needle 906 piercing the balloon) prior to insertion into the NNF-filled balloon 504 such that the needle 906 can then be directly used to inject the hard cement into the balloon 904 after insertion of the combination needle 906/balloon 904 into the NNF-filled balloon 504. In the example of FIG. 11, the balloon 904 has been filled with the cement (e.g., PMMA), which hardens after injection in liquid form, such that the balloon 904 acts as a solid spacer to keep the vertebral bones apart (e.g., such as the vertebral body 102 below and another vertebral body above). Thus, in some embodiments, the cement-filled balloon 904 may have a diameter that is substantially equal to a distance between adjacent vertebral bones. While the inner balloon 904 acts as a solid spacer, the outer NNF-filled balloon 504 acts as a mobile and elastic disc, allowing controlled, slower motion but “firming up” when forces become too great. In some embodiments, a volume ratio of the NNF-filled balloon 504 to the cement-filled balloon 904 is in a range of about 5:1 to 8:1, but other embodiments are possible. After filling the balloon 904 with the cement, the needle 906/cannula 902 may be removed, for example, as shown in FIG. 12. In some cases, the balloon 904 may be made of a thick rubber or similar material that seals once the needle 906/cannula 902 is retracted. In some examples, the balloon 904 may be sealed and detached from the needle 906/cannula 902. In some embodiments, after filling the balloon 904 and removing of the needle 906/cannula 902, the small hole 117 in the AF 112 may be closed or sealed, or simply left to close on its own. Whether the small hole 117 in the AF 112 is sealed or not, the NNF-filled balloon 504 and the cement-filled balloon 904 both remain within the inner disc space (e.g., the cavity 204). It is also noted that in various embodiments the shape of the cement-filled balloon 904 may be roughly spheroidal.

In some examples, the cement-filled balloon 904 and the NNF-filled balloon 504 may be described as a balloon-in-a-balloon assembly (BBA). In the example described above, the BBA may be assembled in vivo, with the interior cement-filled balloon 904 being injected into the NNF-filled balloon 504 following inflation (filling) of the NNF-filled balloon 504. Alternatively, in some cases, the BBA may be pre-assembled, such that both balloons 504/904 (initially deflated) are inserted together into the cavity 204. In this example, and after insertion of the pre-assembled BBA into the cavity 204, the balloons 504 and 904 may then be filled separately or simultaneously. If filled separately, in some cases, the balloon 504 may first be filled with the NNF, and then the balloon 904 may be filled with the cement. Alternatively, in some cases, the balloon 904 may first be filled with the cement, and then the balloon 504 may be filled with the NNF. If filled simultaneously, in some cases, the balloon 504 may be filled with the NNF at the same time that the balloon 904 is filled with the cement.

In some embodiments, an alternative BBA assembly may be formed such that the balloon 504 includes a first NNF and the balloon 904 includes a second NNF different than the first NNF. For instance, the first NNF may exhibit a first change in viscosity (e.g., instantaneous or over time) with applied force, and the second NNF may exhibit a second change in viscosity (instantaneous or over time) with applied force, where the first change in viscosity is different than the second change in viscosity. In addition, in some embodiments, each of the first NNF and the second NNF may include one of a dilatant NNF, a pseudoplastic NNF, a rheopectic NNF, or a thixotropic NNF.

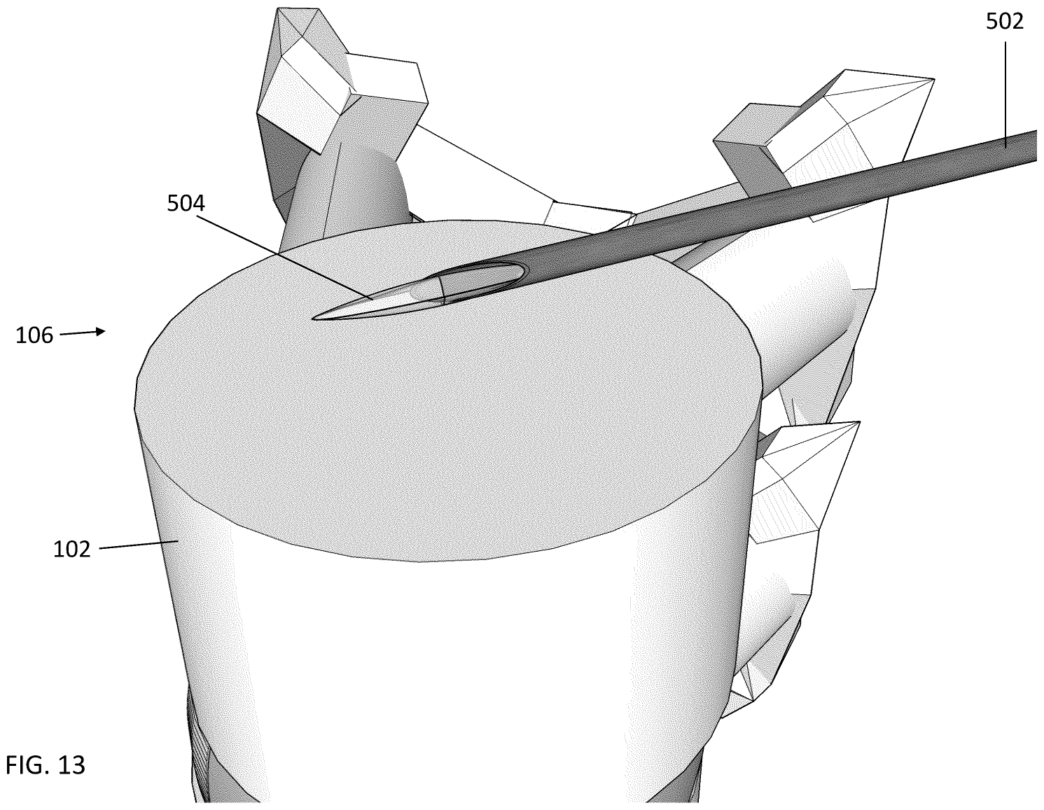

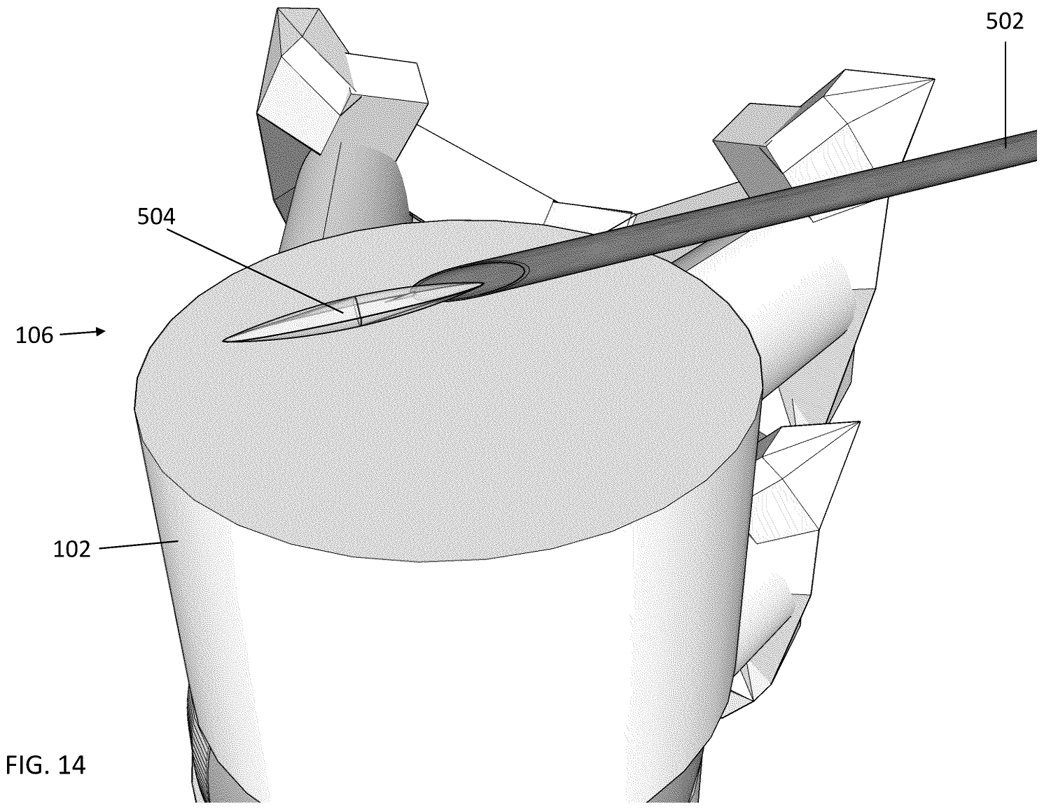

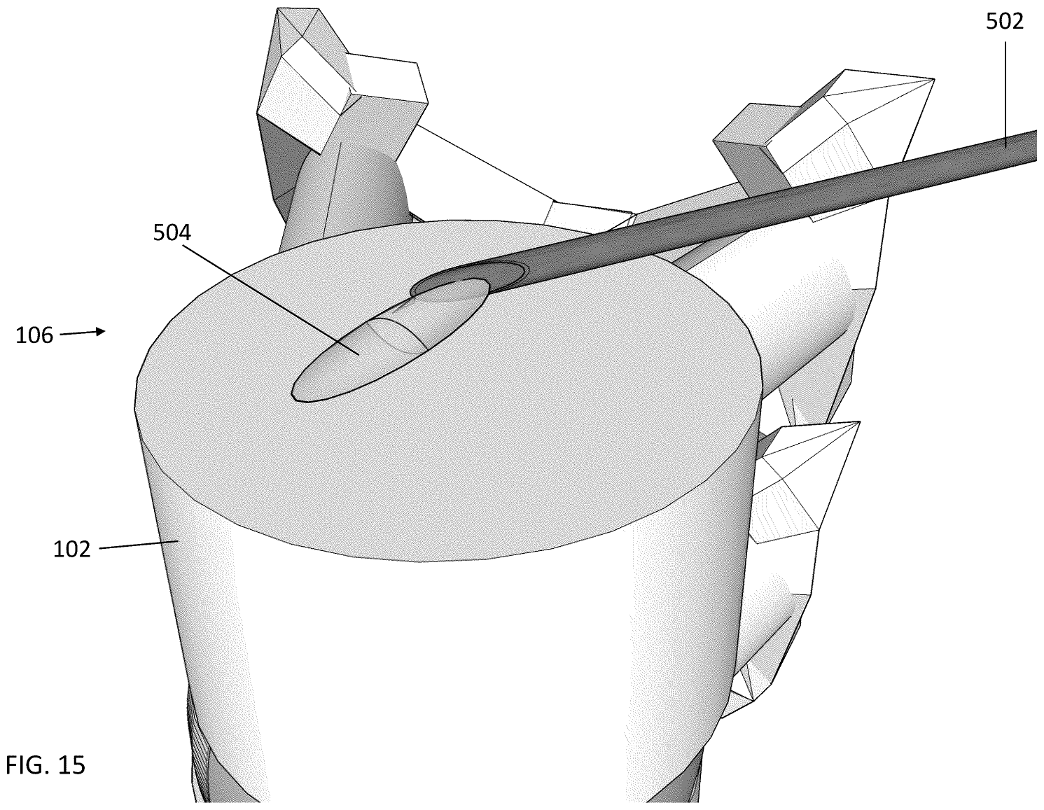

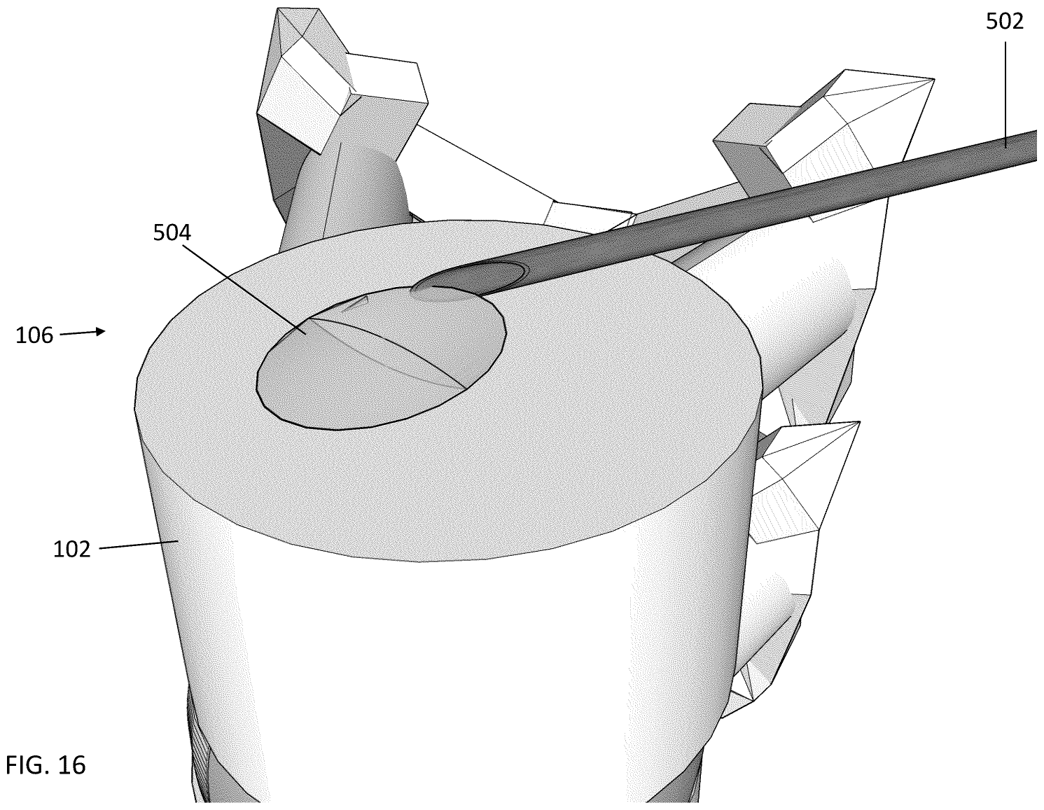

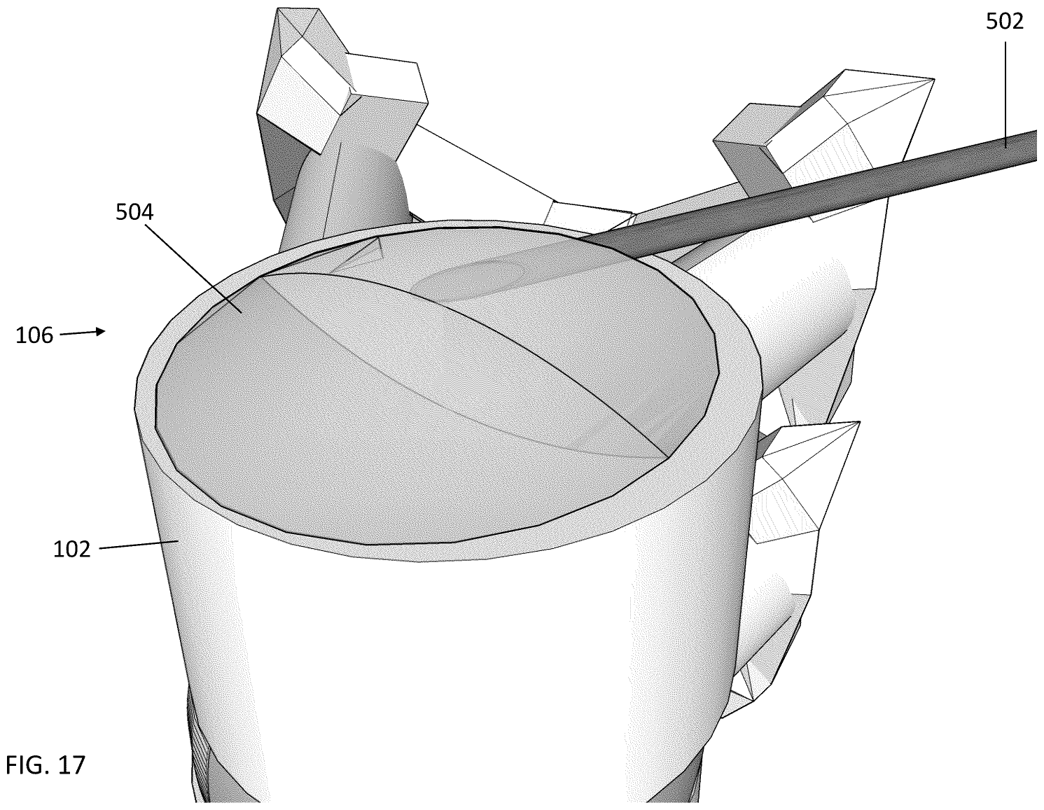

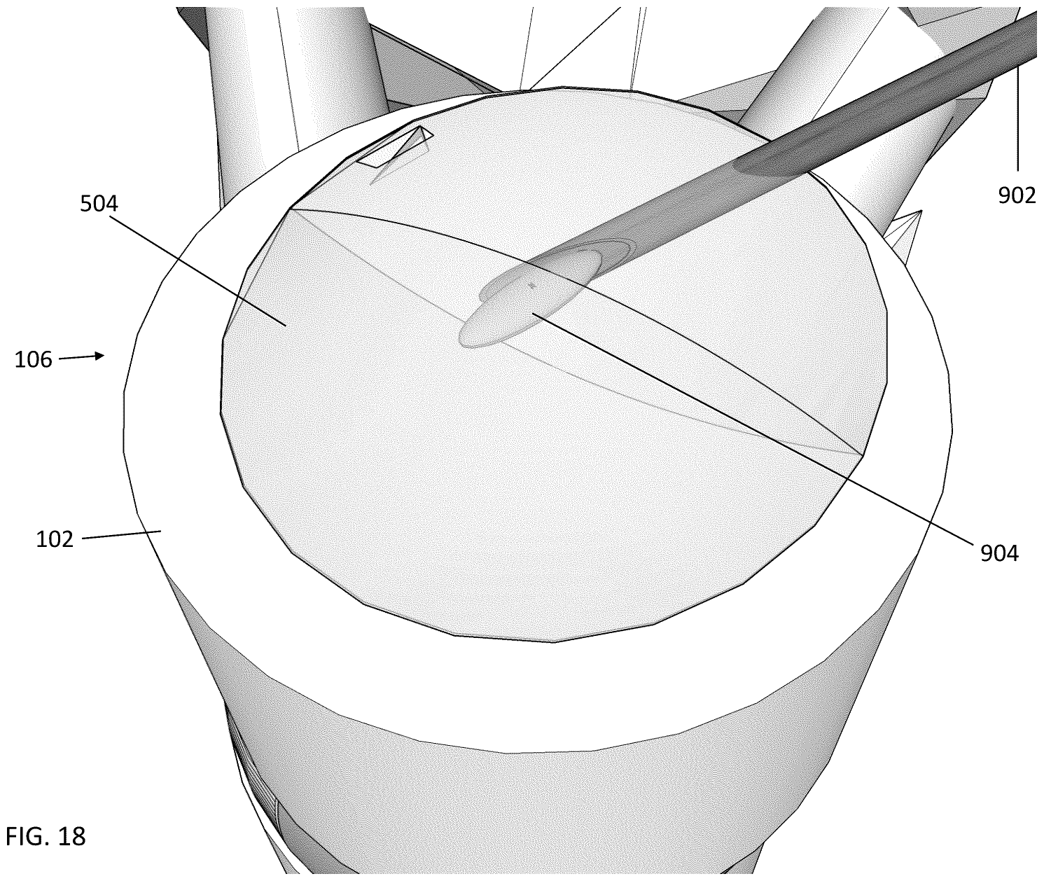

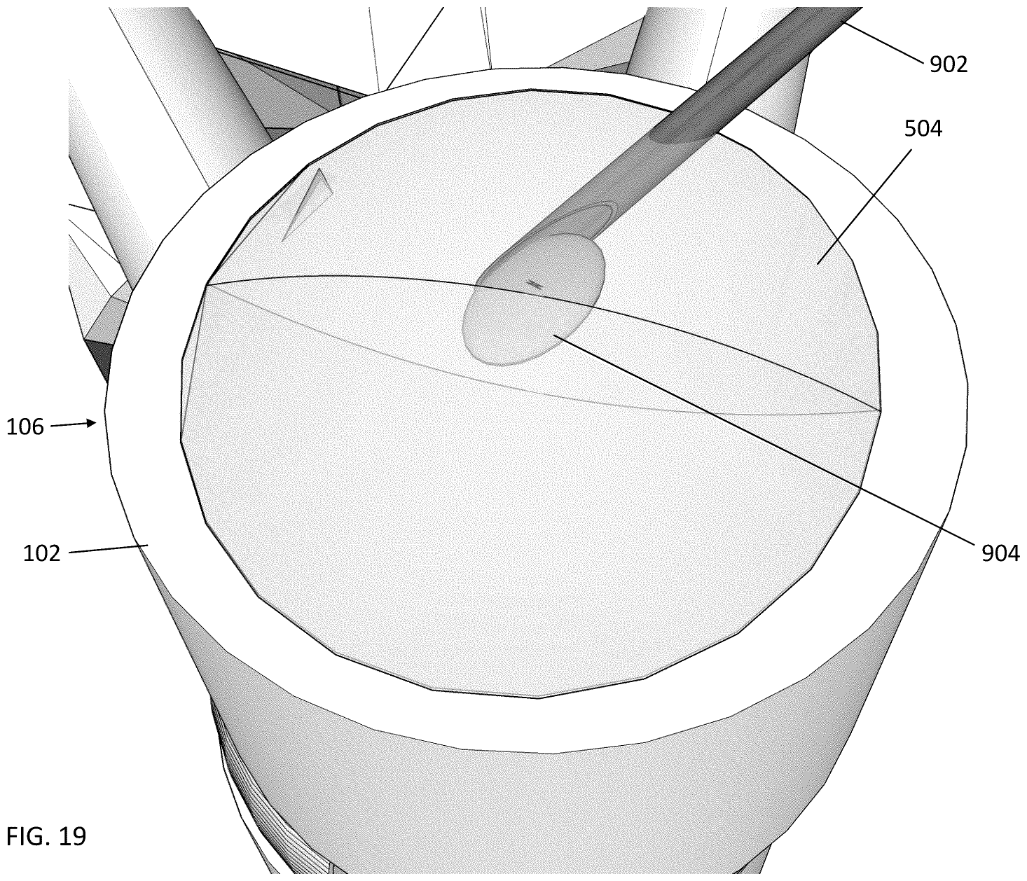

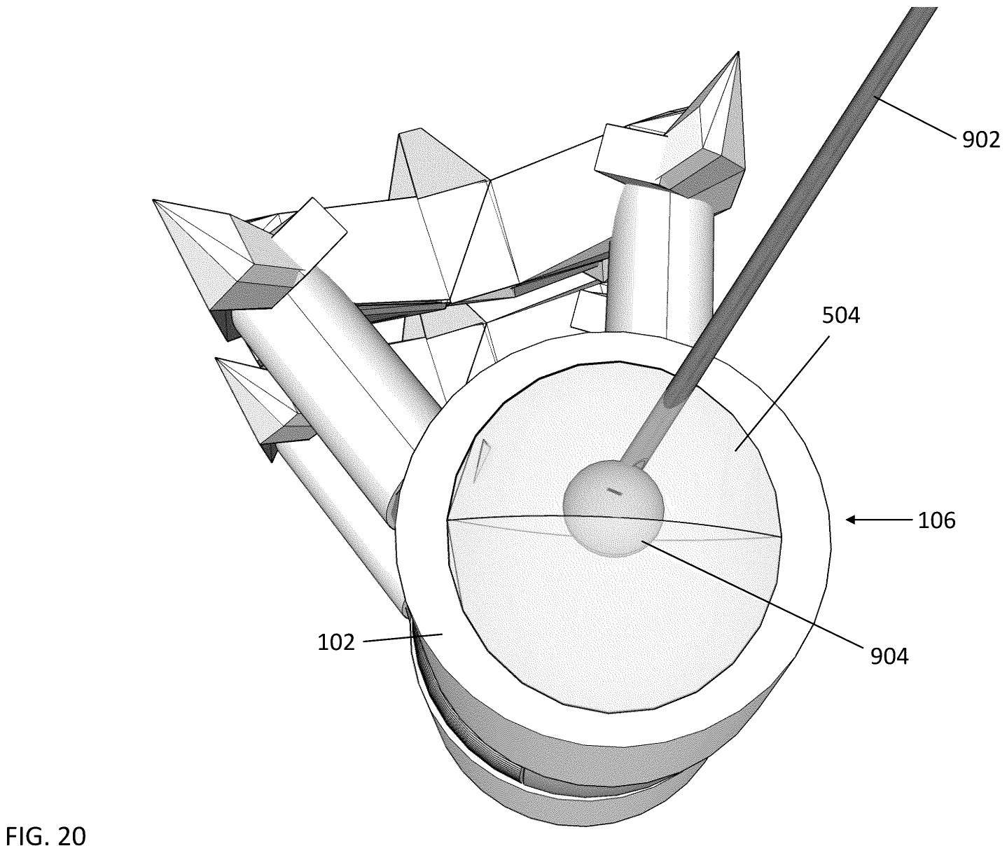

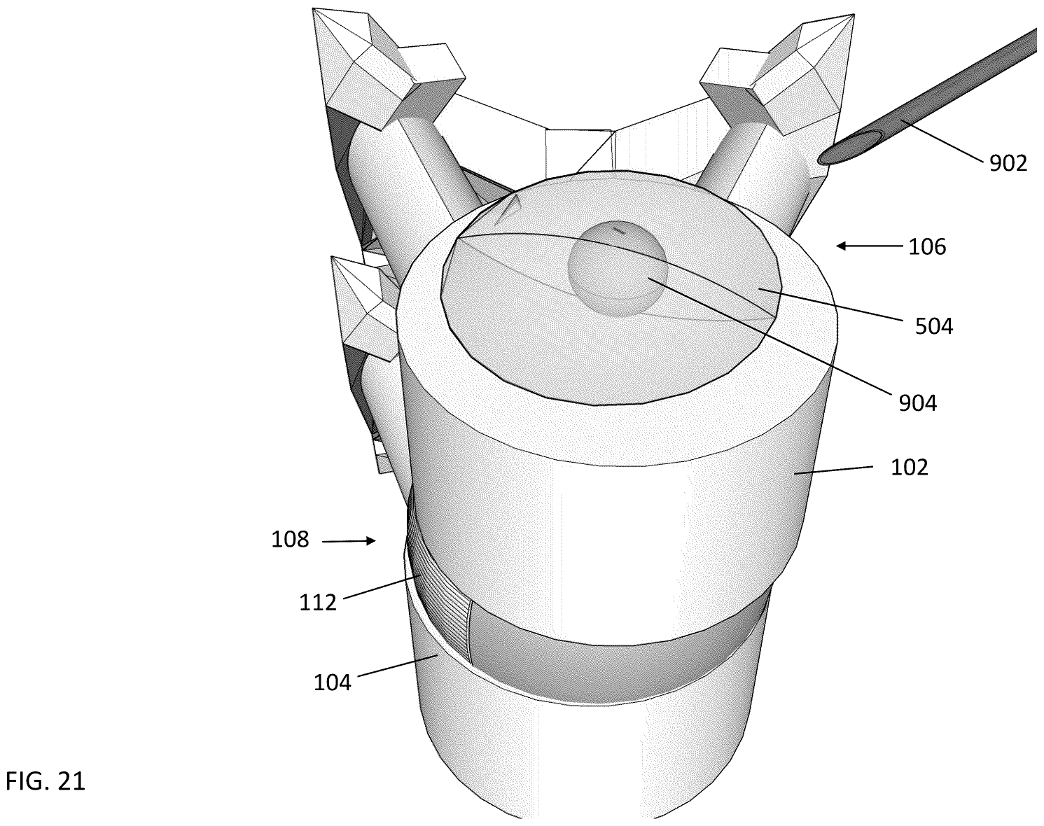

FIGS. 13-21 provide alternative views of the process described above. For example, FIGS. 13-16 illustrate ejection of the balloon 504 from the needle/cannula 502 into the cavity 204 and initial expansion/unfolding of the balloon 504. FIG. 17 illustrates filling of the balloon 504 with an NNF. FIGS. 18-19 illustrate ejection of the balloon 904 from the needle/cannula 902 into the NNF-filled balloon 504 and initial expansion/unfolding of the balloon 904. FIG. 20 illustrates filling of the balloon 904 with a hard cement, and FIG. 21 illustrates removal of the needle/cannula 904 after filling the balloon 904 with the hard cement. In the examples of FIGS. 13-21, and merely for the sake of clarity, the AF 112 is not shown; however, in practice, it will be understood that the AF 112 would be present.

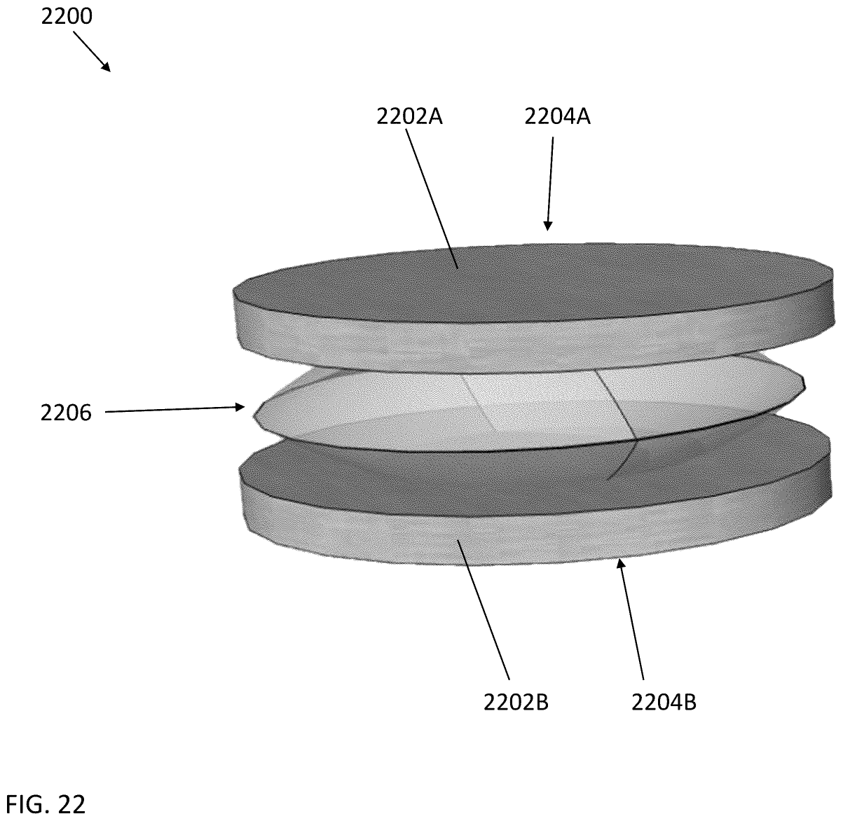

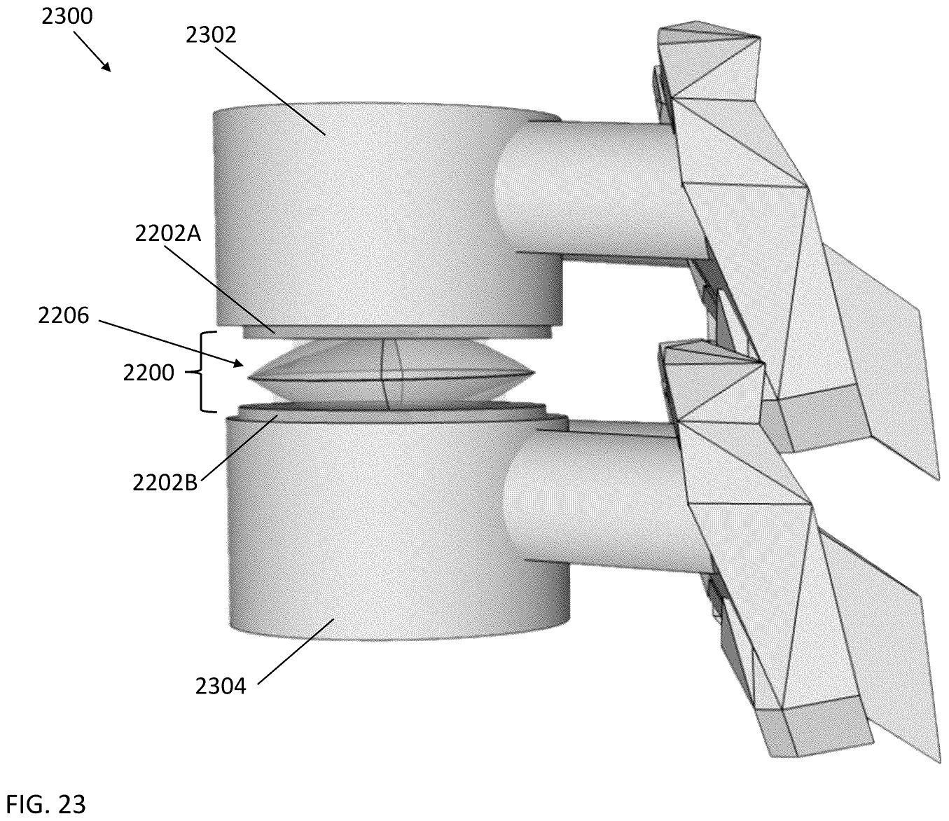

With reference now to FIGS. 22 and 23, illustrated therein are alternative embodiments, in accordance with various aspects of the present disclosure. For instance, while the above discussion focused on a partial disc replacement device including an NNF-based NP replacement device, the examples of FIGS. 22 and 23 are directed to an NNF-based total disc replacement device. FIG. 22 provides a side view of an embodiment of an NNF-based total disc replacement device 2200. The NNF-based total disc replacement device 2200 may include an upper endplate 2202A and a lower endplate 2202B that are configured for attachment to vertebral bodies (e.g., such as the vertebral bodies 102, 104, discussed above) along respective outer surfaces 2204A, 2204B of the upper and lower endplates 2202A, 2202B. In some embodiments, the upper and lower endplates 2202A, 2202B are composed of a metal or metal alloy such as a cobalt chromium molybdenum alloy, a cobalt chromium alloy, a titanium-coated cobalt-chromium metal alloy, a titanium-coated cobalt chromium molybdenum alloy, titanium, a titanium alloy, combinations thereof, or another appropriate metal or metal alloy. In some cases, the upper and lower endplates 2202A, 2202B are composed of an alternative biocompatible metal or plastic. In an embodiment, the upper and lower endplates 2202A, 2202B may include teeth, screw attachments, and/or keels on the respective outer surfaces 2204A, 2204B of the upper and lower endplates 2202A, 2202B, where the outer surfaces 2204A, 2204B attach to the vertebral bodies at a bone-implant interface. In some examples, the upper and lower endplates 2202A, 2202B may attach to the vertebral bodies with bony ingrowth and/or ongrowth surface coating or etching on the surfaces contacting the adjacent vertebral bodies. In some cases, the upper and lower endplates 2202A, 2202B may be coated (e.g., along the respective outer surfaces 2204A, 2204B) with a porous layer of a different metal that encourages bone from the vertebral body to grow onto the endplates 2202A, 2202B, providing extra stability. As shown in FIG. 22, the NNF-based total disc replacement device 2200 further includes an NNF-filled balloon 2206 sandwiched between the upper and lower endplates 2202A, 2202B. In some embodiments, the NNF-filled balloon 2206 may be attached to each of the upper and lower endplates 2202A, 2202B, along respective inner surfaces of the upper and lower endplates 2202A, 2202B, using an adhesive or other alternative binding process. The NNF-filled balloon 2206 may be substantially similar to the NNF-filled balloon 504, discussed above. Thus, in various embodiments, the NNF-filled balloon 2206 may in some cases exclusively include an NNF material, or alternatively the NNF-filled balloon 2206 may also include a solid spacer disposed within the NNF-filled balloon 2206, similar to the cement-filled balloon 904, discussed above. In some examples, the NNF-based total disc replacement device 2200 may also include an artificial AF (not shown) that surrounds the NNF-filled balloon 2206. It will be understood that while some features of the NNF-based total disc replacement device 2200 have been shown and described, these features are not meant to be limiting in any way. Further, it will be understood that the features discussed above with respect to the NNF-based NP replacement device may equally apply, in some embodiments, to the NNF-based total disc replacement device 2200.

FIG. 23 illustrates a lateral view of a portion of vertebrae 2300 of a human spine, similar to the portion of vertebrae 100, discussed above. Thus, the portion of vertebrae 2300 may likewise be part of the lumbar, thoracic, or cervical spine. In particular, the illustrated portion of vertebrae 2300 includes vertebral bodies 2302, 2304. The NNF-based total disc replacement device 2200, including the upper and lower endplates 2202A, 2202B and the NNF-filled balloon 2206, is implanted between the adjacent vertebral bodies 2302, 2304. In some examples, the upper endplate 2202A attaches to the vertebral body 2302 (e.g., via respective outer surface 2204A), and the lower endplate 2202B attaches to the vertebral body 2304 (e.g., via respective outer surface 2204B). Once inserted, the NNF-based total disc replacement device 2200 may serve to restore both the natural distance between the adjacent vertebral bodies 2302, 2304 and the natural motion of the spine. In particular, it is the NNF-filled balloon 2206 that serves as the motion mechanism for the NNF-based total disc replacement device 2200. In various embodiments, the NNF-based total disc replacement device 2200 may be initially inserted between the vertebral bodies 2302, 2304 with the NNF-filled balloon 2206 in a deflated state, a partially inflated state, or a fully inflated state. If inserted in a deflated state or a partially inflated state, the balloon may be subsequently filled (to the extent desired) with an NNF material and (optionally) with a solid spacer, as discussed above.

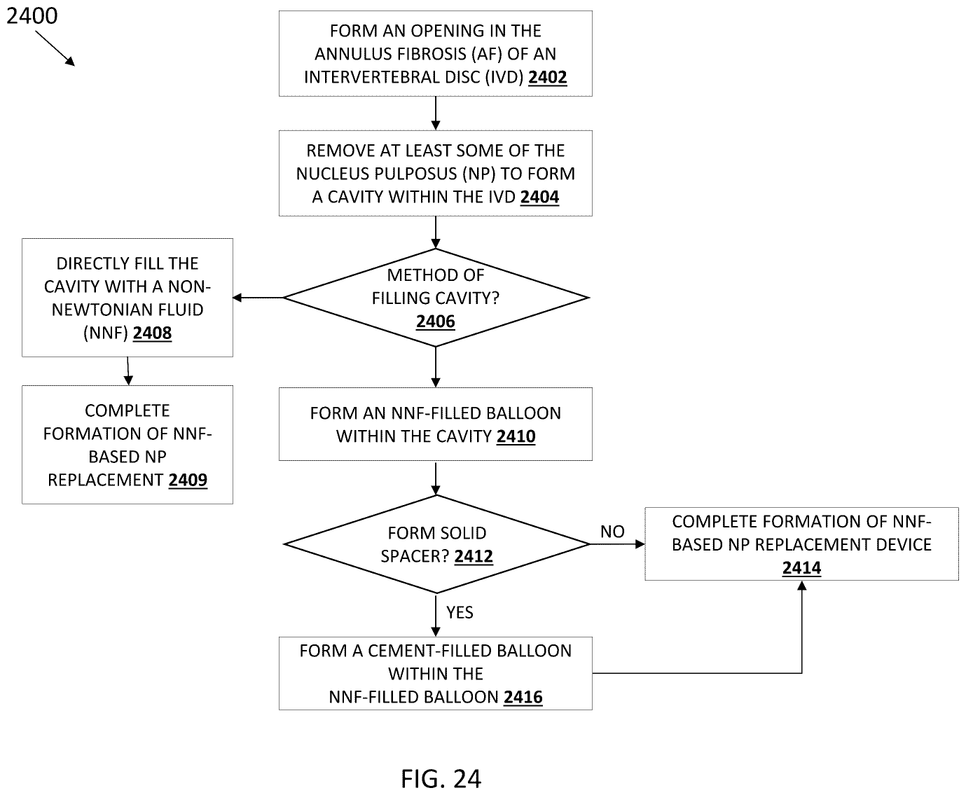

Referring to FIG. 24, illustrated therein is a method 2400 of forming an NNF-based NP replacement device, in accordance with various embodiments. The method 2400 is discussed below with reference to the methods and structures described above. It will be understood that additional steps may be performed before, after, and/or during the method 2400, without departing from the scope of this disclosure. Moreover, it is noted that the process steps of method 2400, including any descriptions given with reference to the figures are merely exemplary and are not intended to be limiting beyond what is specifically recited in the claims that follow.

The method 2400 begins at block 2402 where an opening is formed in the AF of an IVD. In an embodiment of block 2402, and as shown in the example of FIG. 2, a hole 117 may be formed in the AF 112. To be sure, in some embodiments, a blunt cannula may be used to divide, rather than cut, the AF 112. The method proceeds to block 2404 where at least some of the NP 110 is removed to form a cavity 204 within the IVD. In an embodiment of block 2404, and as shown in the examples of FIGS. 3-5, substantially all of the NP 110 may be removed/aspirated. In another embodiment of block 2404, a first portion of the NP 110 may be removed, while a second portion of the NP 110 is unremoved and remains within the IVD. The method proceeds to block 2406 where a method of filling the cavity 204 within the IVD is determined. In one embodiment, the method may proceed from block 2406 to block 2408 where an NNF is freely injected into the cavity 204 (e.g., using a needle/cannula), with the surrounding AF 112, the vertebral body below, and another vertebral body above serving to contain the NNF. After freely injecting the NNF into the cavity 204, the method proceeds to block 2409 to complete formation of the NNF-based NP replacement, which may include removal of a needle/cannula used to fill the cavity with the NNF, closing or sealing the hole 117 in the AF 112, a combination thereof, or other appropriate completion steps. Alternatively, in another embodiment, the method may proceed from block 2406 to block 2410 where an NNF-filled balloon 504 is formed within the cavity 204, as shown in the examples of FIGS. 5-8. After forming the NNF-filled balloon 504, the method proceeds to block 2412 where it is determined whether to form a solid spacer within the NNF-filled balloon 504 (now disposed in the cavity 204). In some examples, it is determined that a solid spacer will not be formed, and the method proceeds from block 2412 to block 2414, to complete the formation of the NNF-based NP replacement device. In some embodiments, completion of the formation of the NNF-based NP replacement device may include removal of a needle/cannula used to fill the balloon 504, sealing and detaching the balloon from the needle/cannula, closing or sealing the hole 117 in the AF 112, a combination thereof, or other appropriate completion steps. In another embodiment, at block 2412 it is determined that a solid spacer will be formed, and the method proceeds from block 2412 to block 2416, where a cement-filled balloon 904 is formed within the NNF-filled balloon 504, as shown in the example of FIGS. 9-12. After forming the cement-filled balloon 904, the method proceeds to block 2414, to complete the formation of the NNF-based NP replacement device, as discussed above.

With respect to the description provided herein, the present disclosure provides an improved disc replacement device and related method that effectively addresses the shortcomings of existing disc replacement devices. In various embodiments, the disclosed disc replacement device includes an NNF-based NP replacement device. Further, various embodiments, the disclosed disc replacement device may include a partial disc replacement device or a total disc replacement device. NP replacement using an NNF-based NP replacement device, in accordance with the various embodiments disclosed herein, may offer several significant advantages over existing NP replacement devices. For instance, NNF-based NP replacement devices are amenable to percutaneous insertion or injection into an intervertebral disc space. In some examples, an NNF may be freely injected into the inner disc space if the AF is intact to contain it. Alternatively, in some cases, the NNF may be inserted through a balloon-catheter type device. In such a balloon insertion method/device, a needle can be placed into the disc space through which remnants of the NP may be aspirated or removed with various manual graspers and/or motorized shavers. Afterwards, an elastic balloon of sufficient thickness may be inserted into a cavity within the disc space that was created when the NP was removed. In various embodiments, the balloon may be filled using an appropriate volume of the NNF to sufficiently fill the NP void (cavity) and sufficiently support the vertebrae above. After filling the balloon with the NNF, the injection needle may be removed from the balloon, which henceforth contains the NNF, or the balloon is then sealed and detached from the cannula. Thereafter, the cannula/tube/needle may be removed while the NNF-filled balloon remains within the inner disc space. In some embodiments, a solid spacer (e.g., such as a cement-filled balloon) may be used in conjunction with the NNF-filled balloon to combine the optimal structural support or separation of the vertebral bodies afforded by the solid spacer, while the surrounding NNF shields the vertebral bony endplates from excessive point loading when sudden forceful loading occurs. Those skilled in the art will recognize other benefits and advantages of the methods and disc replacement device as described herein, and the embodiments described are not meant to be limiting beyond what is specifically recited in the claims that follow.

Thus, one of the embodiments of the present disclosure described a spinal disc replacement device including a first balloon configured for insertion in a cavity within an intervertebral disc (IVD), the cavity surrounded by an annulus fibrosis (AF). In some embodiments, the spinal disc replacement device further includes a first material disposed within the first balloon, where the first material includes a first non-Newtonian fluid (NNF) and provides an NNF-filled balloon within the cavity. In some examples, the NNF-filled balloon is configured to support vertebrae disposed above the IVD.

In another of the embodiments, discussed is a spinal disc replacement device including an upper endplate and a lower endplate configured for attachment to vertebral bodies at a bone-implant interface and along respective outer surfaces of the upper and lower endplates. In some embodiments, the spinal disc replacement device further includes a non-Newtonian fluid (NNF)-filled balloon interposing the upper endplate and the lower endplate, the NNF-filled balloon attached to each of the upper and lower endplates along respective inner surfaces of the upper and lower endplates.

In yet another of the embodiments, discussed is method including forming an opening in an annulus fibrosis (AF) of an intervertebral disc (IVD). In some embodiments, the method further includes removing at least some of a nucleus pulposus (NP) of the IVD, through the hole in the AF, to form a cavity within the IVD. In various examples, the method further includes inserting a first balloon into the cavity through the hole in the AF, and filling the first balloon with a non-Newtonian fluid (NNF).

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A spinal disc replacement device, comprising:
a first balloon configured for insertion in a cavity within an intervertebral disc (IVD), the cavity surrounded by an annulus fibrosis (AF); and
a first material disposed within the first balloon, wherein the first material includes a first non-Newtonian fluid (NNF) and provides an NNF-filled balloon within the cavity;
wherein the NNF-filled balloon is configured to support vertebrae disposed above the IVD.

2. The spinal disc replacement device of claim 1, wherein the cavity is defined by an amount of a nucleus pulposus (NP) removed from the IVD.

3. The spinal disc replacement device of claim 1, wherein a size of the cavity is substantially equal to a volume enclosed by the AF, a first vertebral body beneath the IVD, and a second vertebral body above the AF.

4. The spinal disc replacement device of claim 1, wherein the NNF-filled balloon occupies a substantial entirety of a volume of the cavity.

5. The spinal disc replacement device of claim 1, wherein the NNF-filled balloon has a discoid or oblate spheroidal shape.

6. The spinal disc replacement device of claim 1, wherein the cavity has an arbitrary shape defined at least in part by an amount of a nucleus pulposus (NP) removed from the IVD, and wherein the NNF-filled balloon substantially conforms to the arbitrary shape of the cavity.

7. The spinal disc replacement device of claim 1, wherein the first NNF includes a dilatant NNF.

8. The spinal disc replacement device of claim 1, further comprising:
a second balloon disposed within the NNF-filled balloon; and
a second material disposed within the second balloon to provide a second filled balloon within the NNF-filled balloon;
wherein the second material is different than the first material; and
wherein the second filled balloon provides a solid spacer between adjacent vertebral bones.

9. The spinal disc replacement device of claim 8, wherein the second material includes a bone cement, and wherein the second filled balloon includes a cement-filled balloon.

10. The spinal disc replacement device of claim 9, wherein the bone cement includes polymethylmethacrylate (PMMA).

11. The spinal disc replacement device of claim 8, wherein a volume ratio of the NNF-filled balloon to the second filled balloon is in a range of about 5:1 to 8:1.

12. The spinal disc replacement device of claim 8, wherein the second filled balloon has a spheroidal shape.

13. The spinal disc replacement device of claim 8, wherein the second material includes a second NNF different than the first NNF.

14. A spinal disc replacement device, comprising:
an upper endplate and a lower endplate configured for attachment to vertebral bodies at a bone-implant interface and along respective outer surfaces of the upper and lower endplates; and
a non-Newtonian fluid (NNF)-filled balloon interposing the upper endplate and the lower endplate, the NNF-filled balloon attached to each of the upper and lower endplates along respective inner surfaces of the upper and lower endplates.

15. The spinal disc replacement device of claim 14, wherein the upper and lower endplates are composed of a metal, a metal alloy, a plastic, or a combination thereof.

16. The spinal disc replacement device of claim 14, wherein the upper and lower endplates include teeth, screw attachments, or keels along the respective outer surfaces of the upper and lower endplates.

17. The spinal disc replacement device of claim 14, wherein the upper and lower endplates are coated along the respective outer surfaces of the upper and lower endplates with a porous layer of a different metal that encourages bone from respective ones of the vertebral bodies to grow onto the upper and lower endplates.

18. The spinal disc replacement device of claim 14, further comprising a cement-filled balloon disposed within the NNF-filled balloon.

19. The spinal disc replacement device of claim 18, wherein the cement-filled balloon includes polymethylmethacrylate (PMMA).

20. The spinal disc replacement device of claim 14, further comprising an artificial annulus fibrosis (AF) surrounding the NNF-filled balloon.

21. A method, comprising:
forming an opening in an annulus fibrosis (AF) of an intervertebral disc (IVD);
removing at least some of a nucleus pulposus (NP) of the IVD, through the hole in the AF, to form a cavity within the IVD;
inserting a first balloon into the cavity through the hole in the AF; and
filling the first balloon with a non-Newtonian fluid (NNF).

22. The method of claim 21, wherein a substantial entirety of the NP is removed to form the cavity, and wherein the NNF-filled first balloon occupies an entirety of an available volume of the cavity formed by removal of the substantial entirety of the NP.

23. The method of claim 21, wherein a first portion of the NP is removed to form the cavity while a second portion of the NP remains within the IVD, and wherein the NNF-filled first balloon occupies an entirety of an available volume of the cavity formed by removal of the first portion of the NP.

24. The method of claim 21, further comprising after filling the first balloon with the NNF, inserting a second balloon into the NNF-filled first balloon through the hole in the AF, and filling the second balloon with a bone cement.

25. The method of claim 24, wherein the second balloon filled with the bone cement provides a solid spacer between adjacent vertebral bones.
